(19) United States
(12) Patent Application Publication
(10) Pub. No.: US 2017/0031441 A1
(43) Pub. Date: Feb. 2, 2017

(54) WIRELESS HIGH-DENSITY MICRO-ELECTROCORTICOGRAPHIC DEVICE

(71) Applicant: THE REGENTS OF THE UNIVERSITY OF CALIFORNIA, Oakland, CA (US)

(72) Inventors: Rikky Muller, Kirkland, WA (US); Peter Ledochowitsch, Kensington, CA (US); Hanh-Phuc Le, Richmond, CA (US); Simone Gambini, Southbank (AU); Michel Maharbiz, El Cerrito, CA (US); Jan Rabaey, Berkeley, CA (US)

(73) Assignee: THE REGENTS OF THE UNIVERSITY OF CALIFORNIA, Oakland, CA (US)

(21) Appl. No.: 15/226,502

(22) Filed: Aug. 2, 2016

Related U.S. Application Data
(63) Continuation of application No. PCT/US2015/014905, filed on Feb. 6, 2015.
(60) Provisional application No. 61/937,434, filed on Feb. 7, 2014.

(51) Int. Cl. G06F 3/01; A61B 5/04

(52) U.S. Cl. CPC G06F 3/015; A61B 5/04001

(57) ABSTRACT

A minimally invasive, wireless ECoG microsystem is provided for chronic and stable neural recording. Wireless powering and readout are combined with a dual rectification power management circuitry to simultaneously power to and transmit a continuous stream of data from an implant with a micro ECoG array and an external reader. Area and power reduction techniques in the baseband and wireless subsystem result in over 10×IC area reduction with a simultaneous 3× improvement in power efficiency, enabling a minimally invasive platform for 64-channel recording. The low power consumption of the IC, together with the antenna integration strategy, enables remote powering at 3× below established safety limits, while the small size and flexibility of the implant minimizes the foreign body response.

### CROSS-REFERENCE TO RELATED APPLICATIONS

This application is a 35 U.S.C. §111(a) continuation of PCT international application number PCT/US2015/014905 filed on Feb. 6, 2015, incorporated herein by reference in its entirety, which claims priority to, and the benefit of, U.S. provisional patent application Ser. No. 61/937,434 filed on Feb. 7, 2014, incorporated herein by reference in its entirety. Priority is claimed to each of the foregoing applications.

The above-referenced PCT international application was published as PCT International Publication No. WO 2015/120324 on Aug. 13, 2015, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND

1. Technical Field

This description pertains generally to signal acquisition, and more particularly to neural signal acquisition.

2. Background Discussion

Chronic brain-computer interfaces are an emerging technology that aims at restoring motor or communication function in individuals with spinal cord injuries and/or neurodegenerative diseases. Electrocorticography (ECoG) is a brain-recording modality that utilizes non-penetrating (e.g. sub-dural or epidural electrodes), and shows particular promise for future brain computer interfaces as it can provide similar spatial resolution to more invasive techniques, but reduces scar-tissue formation and hence enables longer-term recordings.

Substantial improvements in neural implant longevity are needed to transition brain-machine interface (BMI) systems from research labs to clinical practice. While action potential (AP) recording through penetrating electrode arrays offers the highest spatial resolution, it comes at the price of tissue scarring, resulting in signal degradation over the course of several months. Electrocorticography (ECoG) is an electrophysiological technique where electrical potentials are recorded from the surface of the cerebral cortex, reducing cortical scarring. However, today's clinical ECoG implants are large, have low spatial resolution (0.4-1 cm) and offer only wired operation.

### BRIEF SUMMARY

An aspect of the present disclosure is a minimally invasive, implantable wireless ECoG microsystem for chronic and stable neural recording. In one embodiment, wireless powering and readout are combined with a micro fabricated antenna and electrode grid that has >10× higher electrode density than clinical ECoG arrays, providing spatial resolution approaching existing penetrating electrodes. Area and power reduction techniques in the baseband and wireless subsystem result in over an order of magnitude in integrated circuit (IC) area reduction, mitigate the need for external discrete components, and provide a simultaneous 3× improvement in power efficiency over existing systems, enabling a minimally invasive platform for 64-channel recording. The low power consumption of the IC, together with the antenna integration strategy, enables remote powering at 3× below established safety limits, while the small size and flexibility of the implant minimizes the foreign body response. The improved implant safety and longevity of the system allows use of wireless ECoG for clinically relevant BMIs.

Another aspect of the present disclosure is a high-density wireless ECoG device configured to be a commercial, fully implantable wireless device, particularly suitable for the animal research market, and once fully vetted, for human-use applications such as detecting or predicting epileptic seizures, providing a readout interface for neuroprosthetic applications, and allowing brain surface recording for monitoring and treating neurological disorders such as Parkinson's disease.

Further aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 shows a schematic system view of the implantable ECoG microsystem in accordance with the present description.

FIG. 2 shows a schematic block diagram of the IC of the implantable ECoG microsystem of FIG. 1.

FIG. 3 shows a detailed schematic diagram of the Gm stage of the front end of FIG. 2.

FIG. 4 shows a detailed schematic diagram of an exemplary embodiment of a front end comprising a simplified schematic of the DAC.

FIG. 5 is a schematic circuit diagram of an exemplary ADC of the front end shown in FIG. 2.

FIG. 6 shows an exemplary voltage waveform at the antenna-IC interface and dual-mode rectifier in accordance with the present description.

FIG. 7 shows an exemplary timing diagram embodying the active rectification scheme of the present description.

FIG. 8 shows a schematic diagram of an active rectifier driver circuitry of the present description.

FIG. 9 shows a schematic circuit diagram of a strong-arm comparator in accordance with the present description.

FIG. 10 shows a plot of the measured closed-loop transfer functions of the ECoG front-end 10 from the electrode input to the ADC output.

FIG. 11 shows a plot of input-referred noise spectral density.

FIG. 12 shows a plot of power rectifier measurements.

FIG. 13 is a plot of wireless transmission bit error rate versus antenna separation.

FIG. 14A shows a recorded waveform of a representative channel and the filtered delta band activity of that waveform plotted together prior to sedative administration.

FIG. 14B shows a recorded waveform of a representative channel and the filtered band activity of that waveform plotted together 15 minutes after sedative (Pentobarbital) administration.

FIG. 14C shows a plot of spectral band power changes for all channels.

FIG. 15 shows a scatter plot of wired and wireless data taken in vivo.

### DETAILED DESCRIPTION

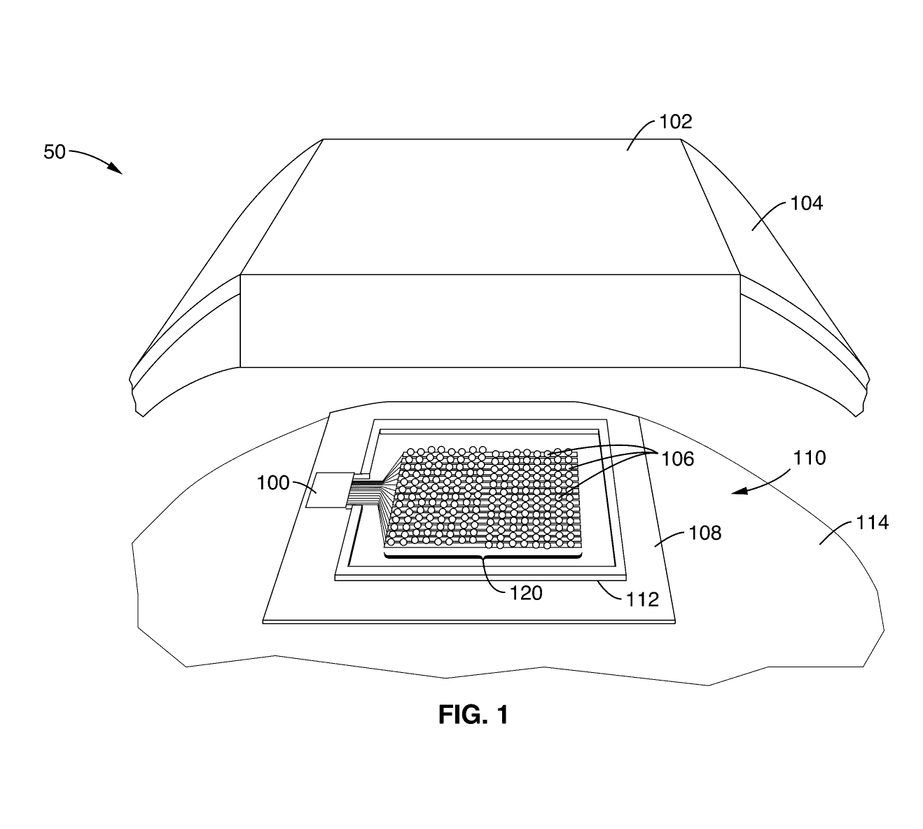

FIG. 1 shows a schematic system view of the implantable ECoG microsystem 50 in accordance with the present description. Microsystem 50 comprises an implant 110 comprising an ECoG grid 120 for neural recordings and control circuitry 100 in the form of an IC configured to interface with and control ECoG grid 120. Implant is shown disposed on cortical surface 114 of the brain, with skull, skin, and intervening tissues between implant and external reader 102 are specifically not shown in FIG. 1 for clarity.

ECoG grid 120 comprises micro-fabricated array of electrodes 106 disposed on a flexible substrate 108, each electrode having sub-mm resolution and configured to acquire and measure signals associated with brain activity.

In a preferred embodiment, entire grid 120 is less than 10 μm thick and sufficiently flexible to conform to the highly folded cortical surface. In one exemplary configuration, the ECoG grid 120 comprises a 4 mm×4 mm, 64-channel array (e.g. 8×8 pattern of electrodes 106). The electrodes have a diameter of 260 μm and an electrode trace spacing of 20 μm. Two reference electrodes (not shown) may be patterned on either side of the array 120 to provide a good spatial average reference, and are sized with 64 times the area of an individual electrode 106 in order to balance the electrode impedances and mitigate 60 Hz noise. In addition, the electrode 106 diameter D_{E} and the electrode edge-to-edge spacing d are configured via the “Spatial Nyquist” condition D_{E}>d/2, acting as a spatial anti-aliasing filter for consistent spatial (spectral) pattern analysis of ECoG activity.

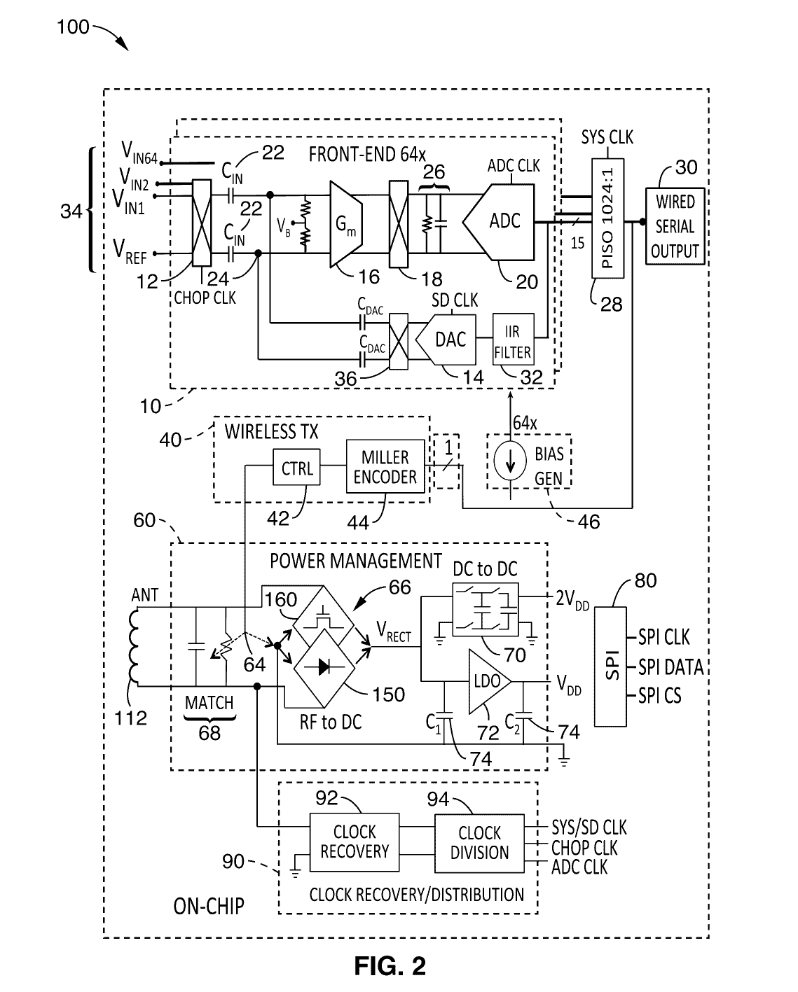

IC 100 comprises an application-specific integrated circuit (ASIC) capable of digitizing the voltage present on the electrodes 106 with power efficiency improvement of more than 3× and an area more than 10× smaller than current state-of-the-art technologies can provide. As will further be detailed below with respect to FIG. 2, the ASIC 100 also integrates circuitry to receive power and to transmit the recorded neural signals wirelessly across the skull, removing the need for percutaneous plugs or ribbon cables.

The implant 110 further includes an antenna 112 that is used to couple wireless power and transmit data wirelessly across the skull. In a preferred embodiment, a single-loop antenna 112 (e.g. 6.5 mm diameter) is monolithically integrated together with the array 120 neural electrodes 106, and is used for both power and data telemetry. This allows for an antenna 112 that is sufficiently large (antenna 112 diameter that is significantly larger than the IC 100) to achieve efficient power coupling, while having micron-scale thinness to provide a high degree of mechanical flexibility to conform to the cortical surface 114 and keeping the implant 110 volume to a minimum. The antenna 112, as shown in FIG. 1, achieves −17.3 dB link gain at 300 MHz while transmitting across a human skull.

A single-loop antenna 112 was chosen for the implant 110 geometry for ease of fabrication with the electrodes 106 in a single mask process. As an example, the ohmic loss in a 250 nm sub-skin-depth conductor is significant, making it favorable to use a single-turn geometry where the conductor length is minimal. The electrode grid 120 dimensions detailed above determined the antenna 112 loop inner diameter of 5.8 mm. A gap is left on one antenna edge for microchip 100 placement and/or routing of the electrode leads 116, as shown in FIG. 1. The loop trace width was optimized to minimize ohmic loss in the sub-skin-depth conductor due to current crowding. For a 250 nm conductor, a width of 0.7 mm degrades the link gain by only 0.5 dB and was therefore chosen for this design. The loss can be further reduced by increasing the width at the expense of metallization and implant area.

System 50 further includes a 1.5 cm diameter external antenna or reader 102, which may be secured to the patients head with band 104, completes the link and powers the implanted electrode array 120 and IC 100, radiating 12 mW of power, 3× lower than the IEEE and FCC recommendation. The external reader 102 also receives backscattered signals, which are decoded into a data stream.

To mitigate the implantation of a large rigid structure, the IC 100 and array 110 of thin-film platinum and gold electrodes 106 are bonded and patterned over a thin-film flexible substrate 108, e.g. a biocompatible polymer such as Parylene C. The highly flexible grid 120 is formed as a thin-film layer (e.g. 10 μm thick) and is sufficiently flexible (Young's modulus E=2.75 GPa) such that it conforms to the cortical folds 114, further reducing neural damage. In a preferred embodiment, platinum black (also a biocompatible material) is electroplated onto the electrode 106 surface to reduce electrode impedance.

The implant 110 may be packaged together via a process such as thermo-compression bonding and thermal annealing to directly connect the integrated circuit to the sensor and to provide a highly biocompatible, hermetic seal.

In one embodiment, the ECoG grid 120 may be fabricated via a wafer-level ECoG fabrication process is performed according to the following steps: 1) Parylene C (polypara-chloro-xylelene, 5 m/layer) is conformally deposited onto a clean silicon carrier wafer; 2) a stack of Pt—Au—Pt (conductor) is electronbeam evaporated and patterned by lift-off; 3) a second layer of Parylene C is deposited as a first insulation layer; and 4) vias are patterned in the parylene by oxygen plasma reactive ion etching (RIE).

The above process flow allows for devices that comprise multiple conductor layers. For device simplicity and robustness, the ECoG electrodes and antenna may be patterned in a single layer by following steps 1 through 4. The following additional steps may be executed to achieve devices with more conductor layers at the expense of complexity: 5) a second conductor layer may be deposited and etched (e.g. second stack of Pt—Au—Pt that is electronbeam evaporated and patterned by lift-off); 6) a third layer of Parylene C is deposited as a second insulation layer; 7) via etch in etch in oxygen plasma, 8) device released in mild detergent and annealed; 9) electroplate PT-black. As a final step, a high-temperature (200 C) anneal may be performed in a nitrogen atmosphere to improve device lifetime.

Bonding of metalized Parylene C devices to rigid structures can present challenges. Conventional solder bonds very poorly to platinum, and even if gold was patterned as the top layer of the metal stack, the metal-Parylene adhesion is not sufficient to robustly support the mechanical load. As a result, anisotropic conductive film (ACF) bonding utilizing a bench-top bonder was adapted to connect high-density ECoG grids with printed circuit boards (PCBs). A similar process can be adapted to perform direct chip-to-flex bonding.

It is further appreciated that while the ECoG grid array disclosed in FIG. 2 is a preferred embodiment, that in practice, this system may be comprised of any micro-scale ECoG grid available in the art.

FIG. 2 shows a schematic block diagram of the IC 100, which includes circuit modules for signal acquisition (e.g. front end 10), a matching network 68, clock recovery 90, communication 40, power management 60, system clock 28 and a bias generator 46.

This IC 100 is optimized for both low power consumption (to minimize the power transmitted by the reader and prolong its battery life) and area occupation. Since the IC 100 is the only rigid component of the system, low area occupation is particularly critical. In this embodiment, no external components other than the antenna and electrodes are utilized, demanding innovative power conversion techniques to minimize the use of energy-storage devices. It is also appreciated that other embodiments may include external components, such as energy storage capacitors, or the like.

IC 100 includes a front end 10 amplifier/digitizer that converts the brain activity picked up by the electrodes 106 to digital signals for further processing and/or transmission. A particular advantage of such a subsystem is to provide low input-referred noise while avoiding excessive loading of the high impedance electrodes, while preventing the large offset associated with the electrode-tissue interface from saturating the electronics.

The 1 kS/s, 15-bit digital outputs 30 from front end 10 are serialized into a 1 Mbps Miller encoded data stream via the Miller encoder 44 of transmission/communication module 40.

Wireless transmission is performed via controller 42 and antenna 112 by modulating the impedance of an on-chip matching network 68 in order to backscatter the incident RF to the external reader 102. The output data stream 30 is Miller-encoded prior to backscattering to minimize the effect of carrier leakage on bit-error rate (BER) in the interrogator.

The back-scattering of the signal is achieved through a shunt-load modulation switch 64. In this configuration, modulation depth is traded off in order to support simultaneous data and power transfer and a dual-mode RF-to-DC rectifier 66 (having passive 150 and active 160 modes) is employed within power management module (PMU) 60 to handle the input voltage variation.

In addition to RF-to-DC conversion via RF-to-DC rectifier 66, the power management unit (PMU) 60 comprises capacitors 74, a low-dropout linear regulator (LDO) 72 and a DC-to-DC converter 70, that provide 0.5 V and 1.0 V to the chip 100, respectively. Clock recovery 92 and division 94 are also implemented in clock recovery/distribution module 90 as part of the wireless subsystem. IC 100 may also include a port 80 (e.g. SPI or the like).

In a preferred embodiment detailed in FIG. 2, the front-end array (i.e. each front end 10 corresponding to each electrode in array 106) comprises a 64-channel front-end array (e.g. for an 8×8 array 106 with an ADC per channel) that dominates the IC power consumption, making a power-efficient design critical. The acquisition of useful ECoG signals involves an input-referred noise of ˜1 ρV over 1-500 Hz, which is achieved in the presence of a large DC offset (up to 10 s of mV) at the electrode-chip interface.

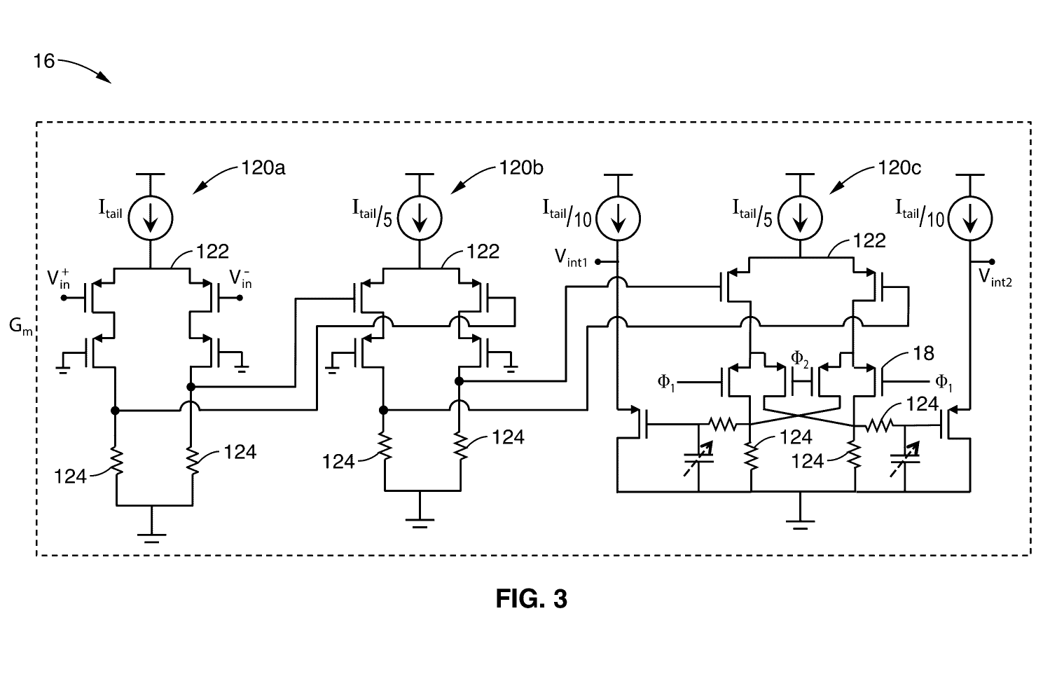

An important feature of the power-efficient configuration of the system 10 of the present description lies in canceling the DC offset early in the signal chain, while minimizing flicker noise. The architecture of front end 10, shown in greater detail in the schematic circuit diagrams of FIG. 3 through FIG. 5, achieves this by using a capacitively coupled chopper stabilized amplifier 16 to minimize 1/f noise, and an oversampled delta-sigma DAC 14 with 15-bit resolution to cancel the upmodulated DC offset.

The ECoG front-end 10 illustrated in FIG. 2 generally comprises a chopper-stabilized, open-loop amplifier. The amplifier comprises input up-modulation chopper switches 12, a Gm stage 16, down-modulation chopper switches 18 and an R-C filter load 26. The output of the amplification stage 34 is connected to an ADC 20.

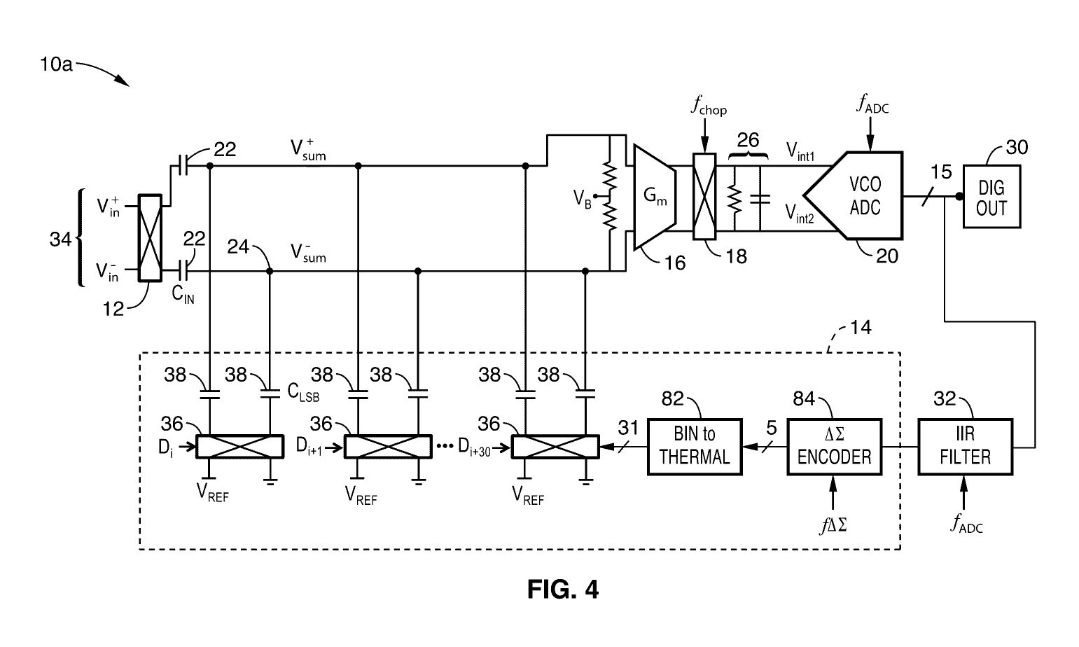

The digital output 30 from amplification stage 34 and ADC 20, which comprises the output of the complete front-end system 10, is then fed back to the input 24 through a digital filter 32 (e.g. an IIR low-pass filter such as an integrator). In preferred embodiment, the digital filter 32 output is then delta-sigma modulated at encoder 84 (see FIG. 4) and fed back to the input through an oversampled capacitor DAC 14. The DAC 14 output is also upmodulated through chopper switches 36 so that the cancellation occurs in the up-modulated signal domain. The summation of the DAC 14 and the input signal (Vᵢₙ⁺, Vᵢₙ⁻) occurs at junction 24 after the input capacitors 22 and the feedback DAC capacitors 38 (see also FIG. 4), at the input of the Gm amplification stage 16.

To prevent instantaneously large amplifier inputs, a DAC is implemented in the feedback path to cancel the upmodulated DC offset. A preferred embodiment of front-end 10 includes five physical DAC bits that are implemented as a 31-element, thermometer coded capacitor array with unit capacitor C_{LSB}. In a preferred configuration, 31C_{LSB}=0.1Cᵢₙ is chosen to cancel the offset while keeping signal attenuation below 1 dB. The large time constants used for filtering the offset are implemented digitally, enabling an area of 0.025 mm² for each front-end. Using an open-loop amplifier improves input impedance, resulting in Zᵢₙ=28 MΩ at 100 Hz with f_{chop}=8 kHz. After chopper demodulation and RC filtering (to suppress ΔΣ noise), the signal is digitized by a pseudo-differential, VCO-based ADC 20 operating at 1 kS/s. The ADC has a raw resolution of 15 bits to suppress quantization noise while processing the ECoG signal, the chopper ripple and the ΔΣ noise the DAC. By designing f_{chop}=Nf_{ADC} (N is an integer) the chopper ripple falls in a notch of the ADC sinc transfer function eliminating the need for a ripple reduction loop.

Referring now to the amplifier 16 shown in the schematic diagram of FIG. 3, the forward path amplification is ideally broadband compared to the signal, at least 1-2 octaves above the Delta-Sigma modulation frequency. In order to achieve more than 3 MHz of bandwidth in approximately 2 μW of power dissipation, three cascaded low-gain stages 120a, 120b, and 120c were used. Each stage is comprised of a PMOS input differential pair 122, a PMOS cascode device to extend the bandwidth by decreasing the miller capacitance at each input gate-drain junction, and a resistive load comprised of polysilicon resistors 124. The polysilicon resistors 124 provide good noise performance and linearity at the cost of die area. Since the amplifier 16 must absorb the large swings of the chopper ripple and Delta-Sigma quantization noise, linearity became a higher priority than die area in this design.

A tunable single pole filter is realized at the output of the third gain 120c stage with the addition of tunable capacitance in parallel with the resistive load 124. The capacitors are realized with NMOS devices in depletion so that they remain linear throughout the signal range. Series resistance is added between the load resistor 124 and the capacitor to reduce the low-pass filter pole without affecting the gain and output swing of the stage 120c. The filter is tunable from a broadband of 3.3 MHz down to 40 kHz. The chopper down-modulation switches 18 are also shown in the gain stage 120c of FIG. 3.

FIG. 4 shows an exemplary embodiment of a front-end 10a comprising a simplified schematic of the DAC 14. To minimize DNL, the DAC 14 is thermometer coded via binary to thermal encoder 82 at the output of the Delta-Sigma encoder 84. To minimize area, each unit capacitor is minimum sized. The capacitors 38 are preferably implemented as metal-insulator-metal (MIM) capacitors that have relatively large minimum dimensions and 5% relative matching, thus maintaining low DNL. In this implementation, V_{REF}=0.5 V and is tied to V_{DD}. To cancel a full-scale voltage of 100 mV, or 50 mV on each differential input, C_{IN}=10C_{DAC}, where C_{DAC}=31C_{LSB}, and C_{LSB}=41 fF. The summation nodes are biased (V_{B}) through a high resistance. The value of this resistance should be high enough such that the high-pass filter pole that it produces together with C_{IN} is well below the lowest chopper frequency and thus out of the signal bandwidth.

Each unit cell of the feedback DAC 14 is comprised of two capacitors 38, C_{LSB}, that are switched in opposite polarity at each phase of the chop clock (not shown). Since the chop clock provides switching at every cycle, the capacitors 38 do not have to be explicitly reset. Thermometer-coded digital control bits, D, control the polarity of each unit cell modulating the amount of charge that is absorbed by the DAC 14 every time the chop clock changes. For example, if there is no offset present at the input, half of the capacitors on V⁺ₛᵤₘ would switch low-to-high, and the other half would switch high-to-low. These capacitors would neutralize and thus not cancel any offset from the input. In reality, since there are 31 unit capacitors, one capacitor should dither between the two states in order to realize zero offset cancellation.

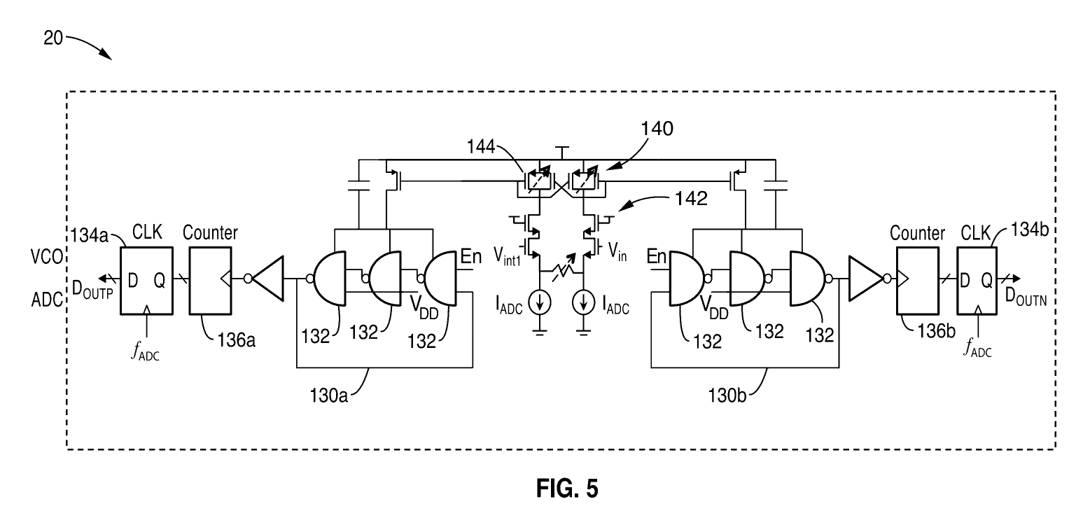

In a preferred embodiment, the ADC 20 comprises a VCO-based ADC. Shown in greater detail in FIG. 5, the positive and negative driver output currents are used as the bias for two single-ended, three-stage CMOS ring oscillators 130a/130b realized with NAND gates 132, which feed the clock 134a/134b inputs of 9-bit digital counters 136a/136b.

VCO-ADC 20 uses a voltage to current converter to drive a ring oscillator 130a/130b, the output of which is fed into a counter 136a/136b for quantization. The quantization sample is taken at every clock cycle and subtracted from the previous quantization level to produce the digital output 30. As shown in FIG. 5, an ADC driver 140 includes a differential-pair V-I converter 142 cascaded with a current-mode programmable gain block 144. The V/I converter load 142 is comprised of nine pairs of unit PMOS devices that can be individually connected either as cross-coupled pairs or as diode-connected devices. When N devices are cross-coupled, the differential mode load impedance seen by the V/I converter equals 1/(9−N)/gₘₚ (N<5 to maintain stability). The outputs of this block are connected to the gates of three matched unit PMOS devices 130a/130b. Changing N can therefore program the differential mode current gain without changing the power dissipation, enabling ease of compensation for varying input amplitudes, which are associated with the distance between the neuron and electrode. Cascode devices are used so that the variable load rather than the drain-source conductance of the input device dominates the gain. Variable degeneration resistors are used to further trade-off gain for linearity.

It is appreciated that front end arrays 10 and 10a shown in FIG. 2 through FIG. 5, while a preferred embodiment, may be substituted with any number of front end/amplification configurations. For example, other front end schemes, such as those detailed in PCT Application. No 2014/51959, filed on Aug. 20, 2014, herein incorporated by reference in its entirety.

Similar to an implantable RFID tag, the wireless subsystem 40 of the ECoG IC 100 uses electromagnetic field backscattering to transmit data. However, rather than using packet-based communication, this system aims to be constantly powered and transmit a continuous stream of data. Architecting the system in this manner avoids the need for large on-chip power and data storage. In order to achieve this, communication modulation depth is traded for matching network 68 impedance that is always finite, and allows power to be rectified continuously. To illustrate this tradeoff, the amplitude of the reflected wave is considered as a function of the matching network resistance and capacitance. The maximum modulation depth occurs when the load Z_{L} is modulated between matched impedance and either an open circuit or a short circuit. However, when the antenna 112 is either in an open or short condition, power cannot be received and rectified. In order to receive power continuously, the system is designed to modulate the impedance of the matching network between a matched condition and finite high impedance. While this results in a lower modulation depth, it allows the incident RF to be received on-chip and be rectified at all times, resulting in continuous-wave power transfer with continuous data modulation.

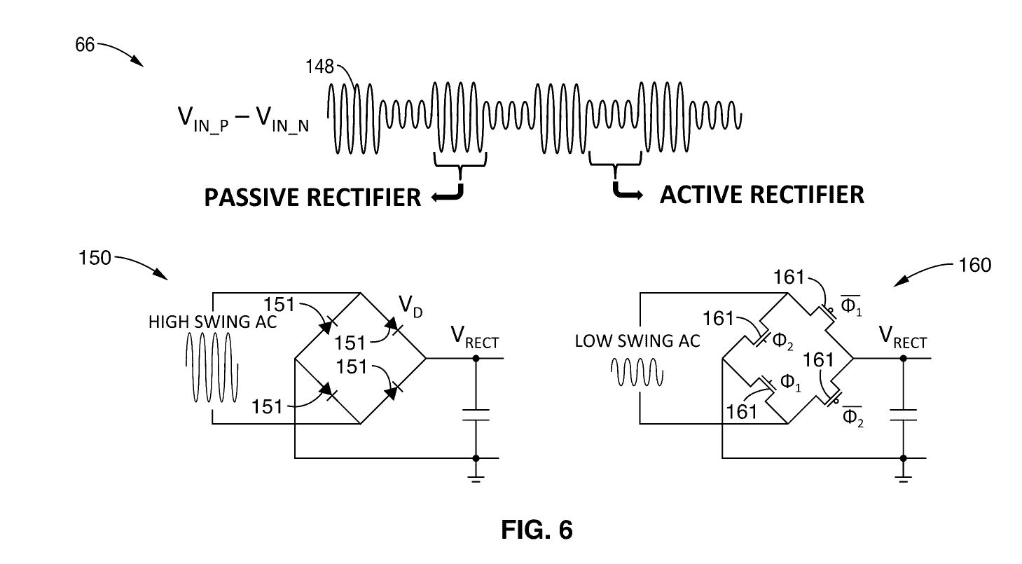

Using the proposed modulation scheme, it is possible to extract power in both matched and unmatched states. However, as illustrated in the signal 148 shown in FIG. 6, the two impedance modulation states lead to large voltage swing variation at the antenna terminals. If such a signal is fed to a conventional rectifier with constant voltage drop, it will cause a big voltage ripple in the output V_{RECT}. If a conventional backscatter modulator is used to transmit 1 MBps data, there will be 1 μs intervals (i.e., corresponding to shorted antenna or symbol “0”) in which no power is collected from the RF and the active circuitry is powered solely by the supply decoupling capacitor. For example, maintaining 1 mV of ripple while drawing 300 μA for 1 μs requires an impractical 300 nF capacitor. On the other hand, if a single rectifier and LDO are used, the LDO would need to attenuate the V_{RECT} ripple of ˜200 mV down to 1 mV, necessitating 40 dB input noise rejection at 1 MHz, which is undesirable in this system due to its power consumption.

In the μECoG IC 100 of the present disclosure, a dual-mode rectifier 66 is used to smooth the voltage at and mitigate the need for a large capacitor or a high-performance LDO. The dual-mode rectifier efficiency (voltage drop) is modulated inversely to the data modulation and therefore the available input power (input voltage swing), in order to maintain a constant output power (constant output voltage at V_{RECT}). This technique reduces the ripple by a factor of 10 at V_{RECT} when compared to a single active rectifier, and is exploited to reduce the supply decoupling capacitance to 4 nF, eliminating the need for external capacitors. If additional supply capacitance is required, it may be added as an external component and packaged together in a hermetic package with the IC.

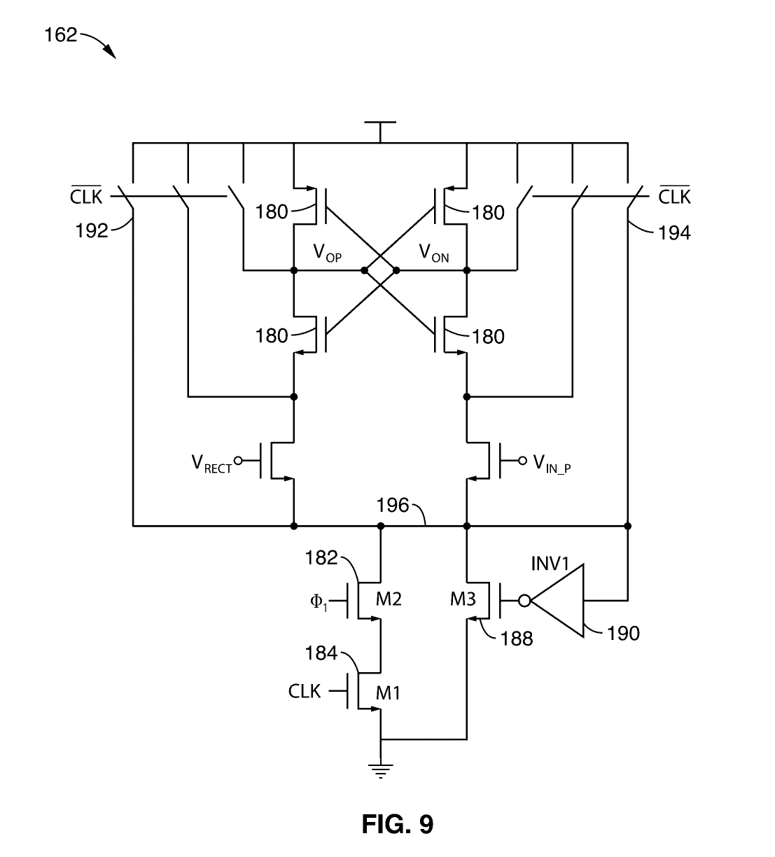

The dual-mode rectifier 66 shown in FIG. 8 is composed of a passive rectifier 150 and an active rectifier 160 connected in parallel. The high-impedance passive rectifier 150 is activated when the data modulated impedance is switched to high impedance, and the low impedance active rectifier 160 is activated when the data modulated impedance is switched to low impedance. Thus, the passive rectification mode operates during the high impedance modulation state when the antenna voltage swing is high. In this mode, the rectifier drops a higher voltage across four diode-connected transistors 151.

During the matched impedance modulation state when the antenna voltage swing is low, the system uses the active rectification mode, implemented with synchronous switches 161 that have small voltage drops. The inverse relationship between input swing and voltage drops over the dual-mode rectifier smoothes the output voltage ripple at V_{RECT} and eliminates the need for a large output capacitance.

In order to reduce ripple by rectifier mode switching, the rectifier 66 voltage drop V_{D} is controlled so that it satisfies the condition V_{LOW-SWING}−V_{D}_{_}_{ACTIVE}=V_{HIGH-SWING}−V_{D}_{_}_{PASSIVE}. In one embodiment, the passive rectifier 150 utilizes diode-connected NMOS transistors 151, whose sizes are scalable with seven binary-coded bits to satisfy the equation above in presence of process variations. Calibration can be automated through an on-chip feedback loop that minimizes V_{RECT} ripple magnitude in real time.

The active rectifier 160 utilizes a mixed-signal feedback loop to control the timing of the synchronous switches and prevent reverse conduction. Since the active rectifier 160 operates on low-input RF voltage, it therefore uses high rectification efficiency, which is achieved using a synchronous switching architecture (switches 161).

Conventionally, in order to control the timing of these power switches, a continuous-time comparator is used to detect the voltage difference across the drain and source of a power transistor. In operation, when V_{IN}_{_}_{P} (or V_{IN}_{_}_{N}) crosses V_{RECT}, the comparator turns the power switch on or off to rectify the input current to the dc load. The power consumption of the comparators is quickly offset by the increased efficiency in applications utilizing a low carrier frequency and high output power.

However, the present system uses rectification at 300 MHz while delivering less than 200 W. While the main power switches operate at 300 MHz, any effort to reduce the switching power of any other circuit is desirable. The self-driven synchronous rectifier 66 shown in FIG. 6 takes advantage of the antenna terminal signals to drive the four transistors, leading to minimal switching power. However, since the turn-on/off timing of the switches depends only on the transistor threshold and the RF input amplitude, this rectifier imposes a specific requirement for the input RF signal to achieve high efficiency. For example, with a RF input, a 0.8 V output, and a 0.5 V threshold, rectifier efficiency can be degraded due to reverse current caused by switch early turn-on at 0.5 V every cycle.

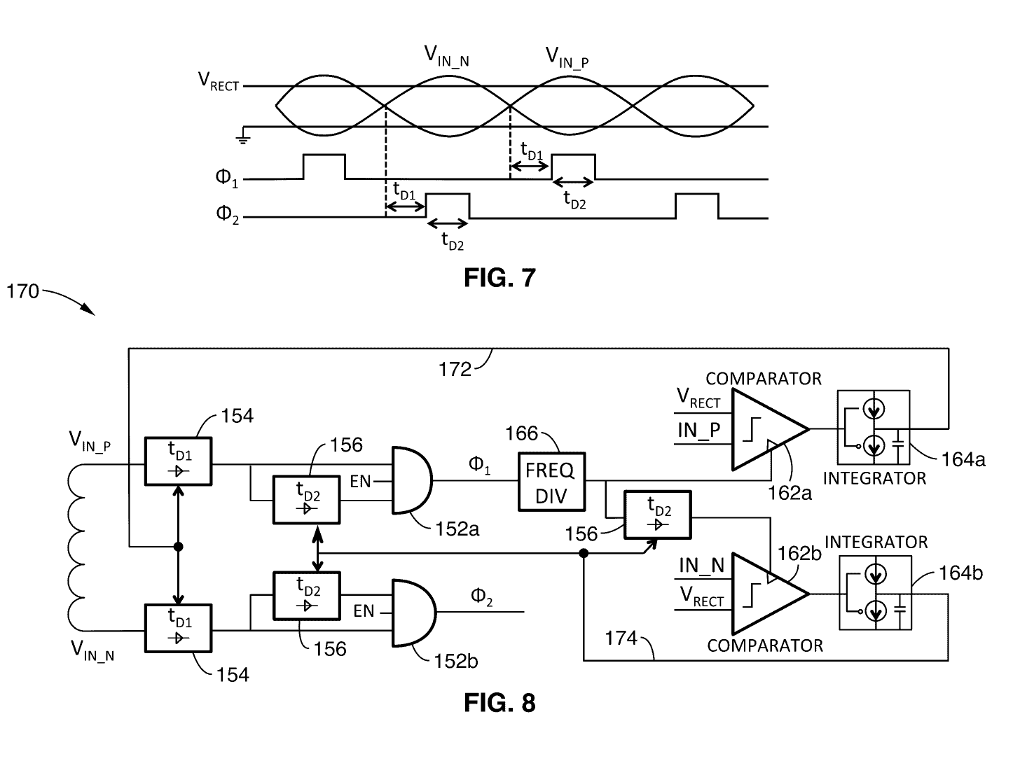

As shown in the timing diagram of FIG. 7 and active rectifier driver circuitry 170 in FIG. 8, the active rectifier 160 utilizes two mixed-signal feedback loops to control the timing of the synchronous switches and prevent reverse conduction. The feedback loops shown in FIG. 8 replace the asynchronous gate-driving comparators of conventional active rectifiers and uses clocked comparators 162a/162b operating at 8× lower frequency than a power carrier, reducing power. A first loop 172 controls the timings of the gate signal including the turn-on delay t_{D1} and the second loop 174 manages the ON period t_{D2}. The turn-on time delay t_{D1} loop 172 comprises a frequency divider 166, current-starved delay cells t_{D1} 154, integrator 164a, φ₁ switch drivers (and-gate 152a) and clocked comparator 162a. The ON period t_{D2} loop comprises a current-starved delay cells t_{D2} 156, integrator 164b, φ₂ switch drivers (and-gate 152b) and clocked comparator 162b. The current-starved delay cell t_{D1} 154 also serves as the RF clock recovery unit. In operation, comparator 162a, triggered by the φ₁ gate signal, detects and controls the t_{D1} loop to zero the difference between V_{IN}_{_}_{P} and V_{RECT} at the turn-on and turn-off instant of the signal. Comparator 162b, triggered by the gate signal φ₁ plus a t_{D2} delay, detects and controls the t_{D2} loop to zero the difference between and at the turn-on and turn-off instant of the signal. Since V_{IN}_{_}_{N} and V_{IN}_{_}_{P} are the same signals with 180° out of phase, the same loop can be used to generate t_{D1} and t_{D2} for the φ₂ signal.

The comparators 162a and 162b are clocked at ⅛th of the carrier frequency and cut the total switching power of the rectifier peripheral circuits by a factor of 3. Although the comparators 162a, 162b are clocked at a slower rate, the switching must still be triggered by the high frequency gate signal in order to have an accurate V_{IN}_{_}_{P}−V_{RECT} cross detection.

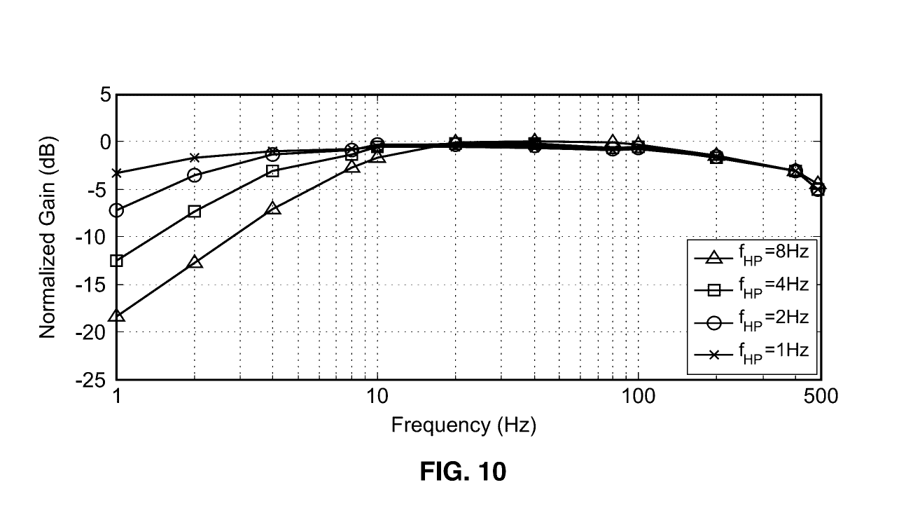

Referring to FIG. 9, accurate cross detection is obtained by using a clock-retimed Strong-Arm comparator 162 architecture, and utilizes retiming of the low frequency comparator clock CLK (162a) or CLK+t_{D2} (162b) to carrier frequency. To enable this feature, the tail clock switch 184 of the Strong-Arm comparator 162 is modified with two series switches M1 184 and M2 182, where M1 is clocked by CLK (for comparator 162a) and CLK+t_{D2} (for comparator 162b), and M2 is driven by the carrier frequency. As shown in the timing diagram in FIG. 7, this configuration allows the comparator to operate at low frequency but to be triggered at the time that the power switches are on. A keeper, comprised of switch M3 188 and inverter INV1 190, is added to the tail node 196 to ensure that the node stays low even as is switching. Pre-charge switches 192, 194 are added to the tail 196 to reset the voltages at each node of the comparator and ensure that the inverter 190 does not sink crowbar current during the pre-charge phase. Cross-coupled inverters 180 are responsible for regeneration in a strong-arm comparator. The outputs of the strong-arm comparator are shown designated by V_{OP} and V_{ON}. There are pre-charge switches that reset the voltages at each node of the comparator.

Electrical characterization of the front-end was performed on a test-PCB with an FPGA interface. All measurements were performed through the full acquisition channels including the on-chip ADCs. Unless otherwise noted, all measurements were performed with _{Fchop}=8 kHz and the low-pass filter bandwidth set to 40 kHz. Post-processing of the digital outputs was performed using MATLAB. Differential sine-wave inputs were produced using a Stanford Research Systems low-distortion signal generator and attenuated to proper input levels at the acquisition channel input.

The test IC was fabricated in a 65 nm 1P7M low-power CMOS process. The total chip area is pad-limited to 2.4 mm by 2.4 mm and the active circuit area totals 1.72 mm², with 1.6 mm² occupied by the front-end array. The total power dissipation of the chip is 225 μW, including the 60% power conversion efficiency.

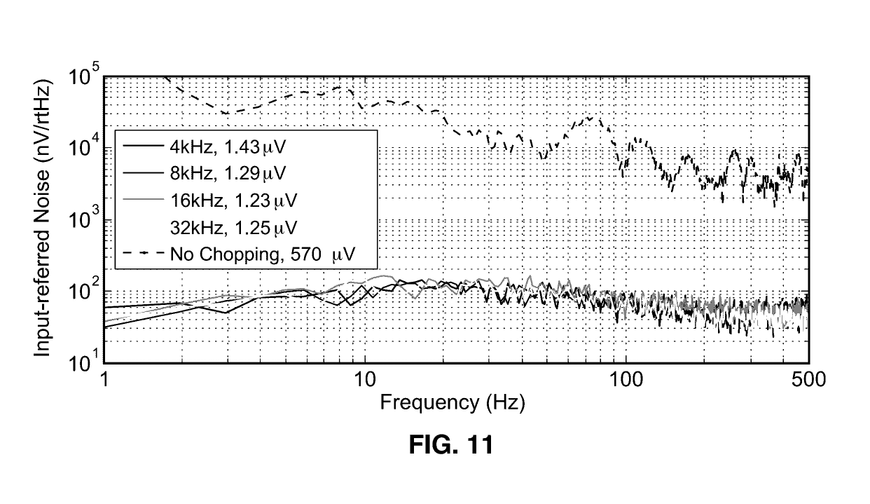

FIG. 10 shows a plot of the measured closed-loop transfer functions of the ECoG front-end 10 from the electrode input to the ADC output. The first-order high-pass pole frequencies are digitally configurable with four such configurations shown. The high-frequency roll-off is due to the sinc transfer function of the ADC.

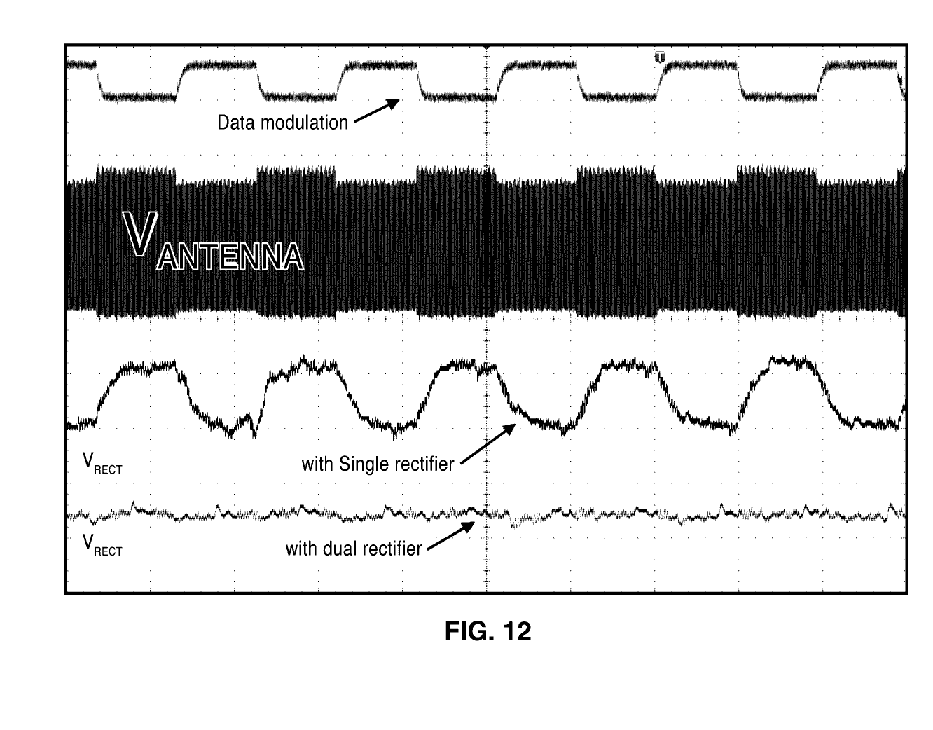

FIG. 11 shows a plot of input-referred noise spectral density, with chopping disabled and for a range of digitally configurable chopper frequencies (and therefore also input impedance). Integrated over 500 Hz, chopper stabilization decreases the noise floor by 400×. Comparing this design against state-of-the-art noise and power efficient ECoG and EEG front-ends, the proposed techniques enabled a 16× area reduction and a 3× improvement in PEF while integrating an ADC per channel.

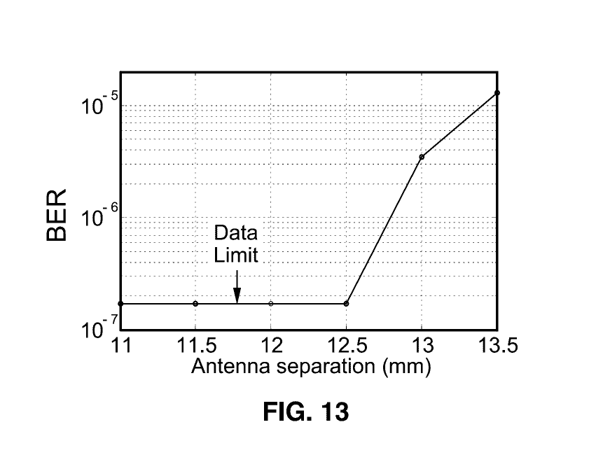

FIG. 12 shows a plot of power rectifier measurements, and in particular, the voltage waveform at the rectifier output attenuated ˜20×. The measurement demonstrates that switching from using a single active rectifier to using a dual-rectifier 66 reduces output voltage (V_{RECT}) fluctuation by ten times. The PMU 60 delivers 160.2 W from 225 W from the implant antenna. The 70% total efficiency is the series combination of the dual rectifier 66 (84% efficient) and LDO 72 (82.5% efficient).

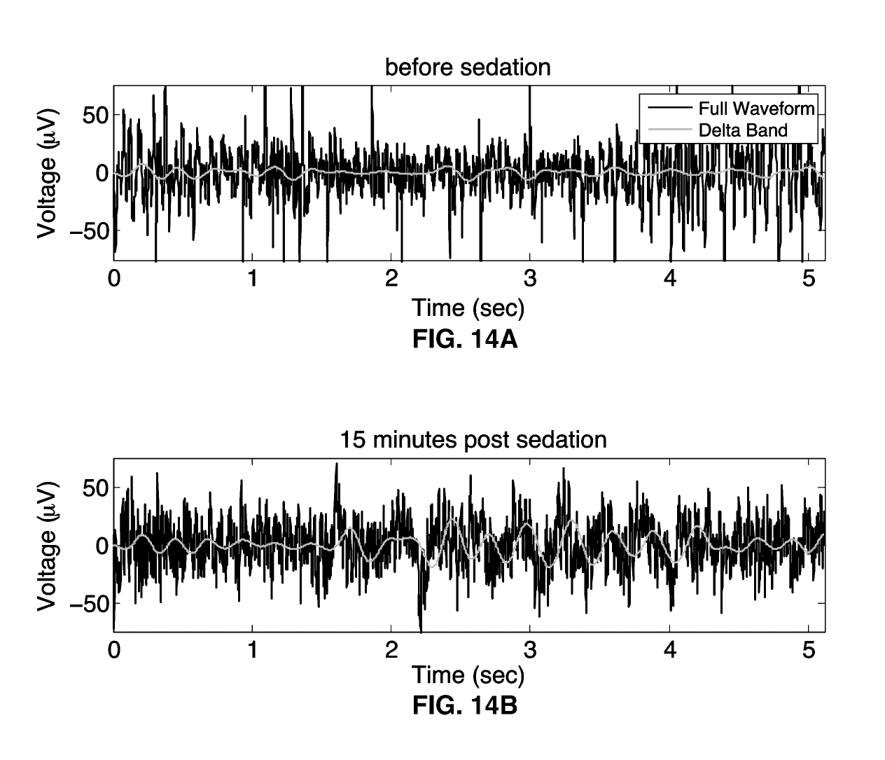

FIG. 13 is a plot of wireless transmission bit error rate (BER) versus antenna separation. In FIG. 13, the performance of the wireless link is verified by wirelessly transmitting a PRBS-7 data pattern generated on-die. Zero errors were found in 5.9 Mb of data resulting in a BER <1.7e-7 with 1 cm antenna separation in air and in vivo. Robustness of the link was verified by varying the link distance and zero errors were found up to 12.5 mm in air.

The performance of the ECoG front-end of the present disclosure was compared to state-of-the-art designs from industry and academic researchers. State-of-the-art noise efficiency is achieved, and, together with a reduced power supply, this work achieves the lowest reported PEF, three times lower than existing systems. The small area (0.025 mm²/ch) enables the highest degree of integration achieved to date in low-frequency high-precision bio-signal acquisition with a 64-channel array in only 1.6 mm of active silicon area and no external components required.

The IC of the present disclosure was assembled together with the micro fabricated ECoG electrodes and antenna on a PCB and implanted in an anesthetized Long-Evans rat over the left cortical hemisphere. Electrical recordings were made on all channels prior to and 15 minutes after the administration of Pentobarbital, a sedative. It is known that anesthesia causes increased δ band (1-4 Hz) oscillations and depressed high-γ (65-125 Hz) activity.

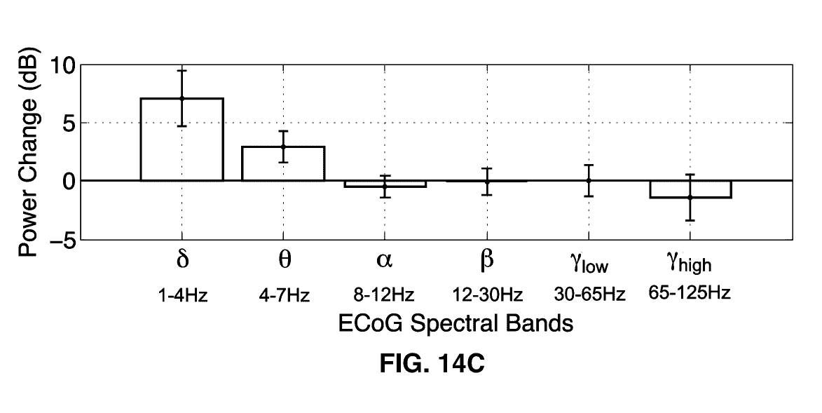

FIG. 14A through FIG. 14C show plots of in vivo system measurement results. FIG. 14A shows a recorded waveform of a representative channel and the filtered band activity of that waveform plotted together prior to sedative administration. FIG. 14B shows a recorded waveform of a representative channel and the filtered band activity of that waveform plotted together 15 minutes after sedative (Pentobarbital) administration. FIG. 14C shows a plot of spectral band power changes for all channels.

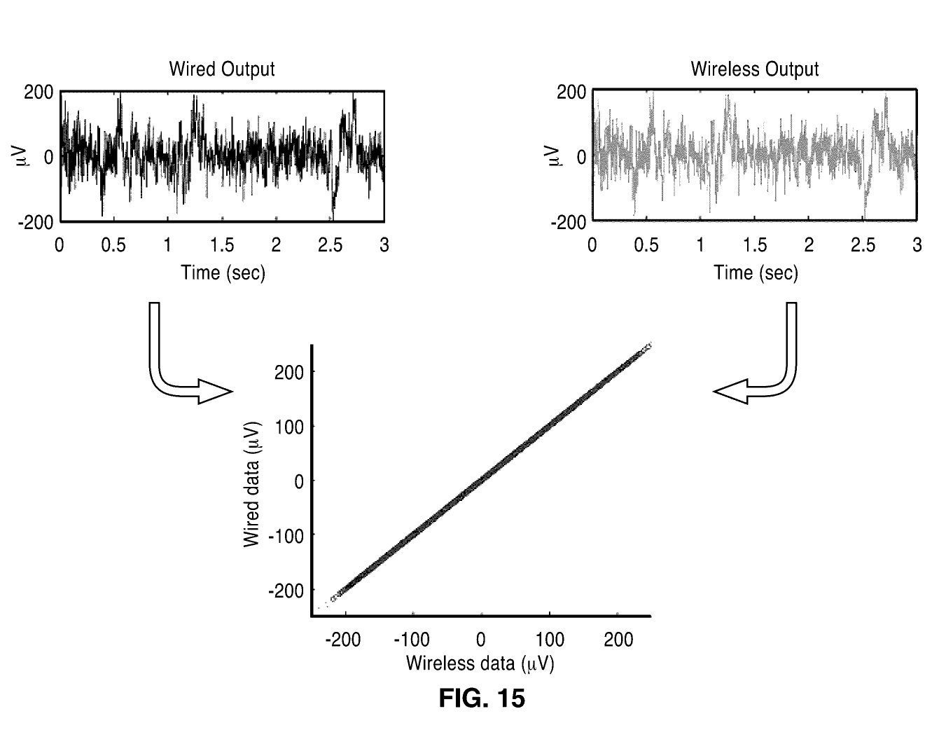

With the entire system active and wirelessly powered, cortical surface potentials from all electrodes were recorded simultaneously through the wired readout and through the wireless link, limiting the data capture length to 3 Mb. FIG. 15 shows a scatter plot of wired and wireless data taken in vivo. As shown in FIG. 15, the two data sets plotted against each other show zero errors in over 3 Mb of data.

While the ECoG system and methods described above are particularly detailed with respect to acquiring and recording neural signals. However, it is appreciated that the electrode array 120 and IC 100 may be configured to be implanted and transmit to and be powered from an external reader from any region of the body where it would be beneficial to have a small-platform, thin-film array for continuously and simultaneously powering and transferring data from the array.

Embodiments of the present technology may be described with reference to flowchart illustrations of methods and systems according to embodiments of the technology, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that the terms “programming” or “program executable” as used herein refer to one or more instructions that can be executed by a processor to perform a function as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors. It will further be appreciated that as used herein, that the terms processor, computer processor, central processing unit (CPU), and computer are used synonymously to denote a device capable of executing the instructions and communicating with input/output interfaces and/or peripheral devices.

From the description herein, it will be appreciated that that the present disclosure encompasses multiple embodiments which include, but are not limited to, the following:

1. A wireless μECoG system, comprising: a micro-fabricated ECoG array of electrodes configured to be implanted at a brain surface to acquire neural signals; an application-specific integrated circuit (IC) coupled to the array of electrodes and configured to record voltages present on the array of electrodes; an antenna coupled to the IC; and an external reader; wherein the IC and antenna are configured to wirelessly transmit a continuous stream of data with the external reader by electromagnetic field backscattering of a signal comprising said data; wherein said continuous stream of data is associated with acquired neural signals from the array of electrodes; and wherein the IC and antenna are configured to be simultaneously and continuously powered by the external reader while wirelessly transmitting the continuous stream of data with the external reader.

2. The system of any preceding embodiment, wherein the array of electrodes and antenna are disposed on a flexible substrate, and have a total thickness of 1 μm to 100 μm.

3. The system of any preceding embodiment, wherein the array of electrodes and antenna are sufficiently flexible to conform to a highly folded cortical surface.

4. The system of any preceding embodiment, wherein the IC is configured to backscatter the signal by modulating the impedance of an on-chip matching network of the IC.

5. The system of any preceding embodiment, wherein the signal comprises an RF signal.

6. The system of any preceding embodiment, wherein the IC comprises a dual-mode rectifier to maintain a constant output power to the IC and array of electrodes while modulating the continuous stream of data for transmission.

7. The system of any preceding embodiment, wherein the dual-mode rectifier comprises a passive rectifier and an active rectifier.

8. The system of any preceding embodiment: wherein the passive rectifier and active rectifier are connected in parallel; wherein the passive rectifier comprises a high-impedance rectifier that is activated when a data modulated impedance is switched to high impedance; and wherein the active rectifier comprises a low impedance active rectifier that is activated when the data modulated impedance is switched to low impedance.

9. The system of any preceding embodiment, wherein the dual-mode rectifier comprises: a passive rectification mode that operates during a high impedance modulation state when a voltage swing of the antenna is high; and an active rectification mode that operates during a low impedance modulation state when a voltage swing of the antenna is low.

10. The system of any preceding embodiment, wherein the active rectification mode is applied with an active rectifier having synchronous switches with small voltage drops.

11. The system of any preceding embodiment, wherein the passive rectification mode is applied with a passive rectifier that drops a higher voltage across a plurality of diode-connected transistors.

12. The system of any preceding embodiment, wherein the IC comprises a shunt-load modulation switch to back-scatter the signal.

13. The system of any preceding embodiment, wherein the IC further comprises a Miller encoder to Miller-encode the continuous data stream prior to backscattering.

14. A wireless μECoG device, comprising: a micro-fabricated, ECoG array of electrodes configured to be implanted at a brain surface to acquire neural signals; an application-specific integrated circuit (IC) coupled to the array of electrodes and configured to digitize a voltage present on the array of electrodes; and an antenna coupled to the IC; wherein the IC and antenna are configured to wirelessly transmit a continuous stream of data with an external reader by electromagnetic field backscattering of a signal comprising said data; wherein said continuous stream of data is associated with acquired neural signals from the array of electrodes; and wherein the IC and antenna are configured to be simultaneously and continuously powered by the external reader while wirelessly transmitting the continuous stream of data with the external reader.

15. The device of any preceding embodiment, wherein the array of electrodes and antenna are disposed on a flexible substrate, and have a total thickness of 1 μm to 100 μm.

16. The device of any preceding embodiment, wherein the array of electrodes and antenna are sufficiently flexible to conform to a highly folded cortical surface.

17. The device of any preceding embodiment, wherein the IC comprises a dual-mode rectifier to maintain a constant output power to the IC and array of electrodes while modulating the continuous stream of data for transmission.

18. The device of any preceding embodiment, wherein the dual-mode rectifier comprises a passive rectifier and an active rectifier connected in parallel; wherein the passive rectifier comprises a high-impedance rectifier that is activated when a data modulated impedance is switched to high impedance; and wherein the active rectifier comprises a low impedance active rectifier that is activated when the data modulated impedance is switched to low impedance.

19. The device of any preceding embodiment, wherein the dual-mode rectifier comprises: a passive rectification mode that operates during a high impedance modulation state when a voltage swing of the antenna is high; and an active rectification mode that operates during a low impedance modulation state when a voltage swing of the antenna is low.

20. The device of any preceding embodiment, wherein the active rectification mode is applied with an active rectifier having synchronous switches with small voltage drops.

21. The device of any preceding embodiment, wherein the passive rectification mode is applied with a passive rectifier that drops a higher voltage across a plurality of diode-connected transistors.

22. A method for wirelessly transmitting μECoG signal across a tissue, comprising: implanting an ECoG array of electrodes at a brain surface; wirelessly digitizing a voltage present on the array of electrodes; acquiring continuous stream of data corresponding to neural signals from the array of electrodes; and backscattering a signal comprising said continuous stream of acquired data and wirelessly transmitting said signal to an external reader; wherein the implant is simultaneously and continuously powered by the external reader while wirelessly transmitting the continuous stream of data with the external reader.

23. The method of any preceding embodiment, wherein the array of electrodes are disposed on a substrate with an antenna, and are sufficiently flexible to conform to a highly folded cortical surface of the brain surface.

24. The method of any preceding embodiment, wherein a constant output power is maintained to array of electrodes while modulating the continuous stream of data for transmission.

25. The method of any preceding embodiment, wherein the continuous stream of data and power transmission are modulated via a dual-mode rectifier.

26. The method of any preceding embodiment, wherein the dual-mode rectifier comprises: a passive rectification mode that operates during a high impedance modulation state when a voltage swing of the antenna is high; and an active rectification mode that operates during a low impedance modulation state when a voltage swing of the antenna is low.

27. The method of any preceding embodiment, wherein the active rectification mode is applied with an active rectifier having synchronous switches with small voltage drops.

28. The method of any preceding embodiment, wherein the passive rectification mode is applied with a passive rectifier that drops a higher voltage across a plurality of diode-connected transistors.

29. The method of any preceding embodiment, further comprising Miller-encoding the continuous data stream prior to backscattering.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A wireless μECoG system, comprising:
a micro-fabricated ECoG array of electrodes configured to be implanted at a brain surface to acquire neural signals;
an application-specific integrated circuit (IC) coupled to the array of electrodes and configured to record voltages present on the array of electrodes;
an antenna coupled to the IC; and
an external reader;
wherein the IC and antenna are configured to wirelessly transmit a continuous stream of data with the external reader by electromagnetic field backscattering of a signal comprising said data;
wherein said continuous stream of data is associated with acquired neural signals from the array of electrodes; and
wherein the IC and antenna are configured to be simultaneously and continuously powered by the external reader while wirelessly transmitting the continuous stream of data with the external reader.

2. A system as recited in claim 1, wherein the array of electrodes and antenna are disposed on a flexible substrate, and have a total thickness of 1 μm to 100 μm.

3. A system as recited in claim 2, wherein the array of electrodes and antenna are sufficiently flexible to conform to a highly folded cortical surface.

4. A system as recited in claim 1, wherein the IC is configured to backscatter the signal by modulating the impedance of an on-chip matching network of the IC.

5. A system as recited in claim 4, wherein the signal comprises an RF signal.

6. A system as recited in claim 1, wherein the IC comprises a dual-mode rectifier to maintain a constant output power to the IC and array of electrodes while modulating the continuous stream of data for transmission.

7. A system as recited in claim 6, wherein the dual-mode rectifier comprises a passive rectifier and an active rectifier.

8. A system as recited in claim 7:
wherein the passive rectifier and active rectifier are connected in parallel;
wherein the passive rectifier comprises a high-impedance rectifier that is activated when a data modulated impedance is switched to high impedance; and
wherein the active rectifier comprises a low impedance active rectifier that is activated when the data modulated impedance is switched to low impedance.

9. A system as recited in claim 6, wherein the dual-mode rectifier comprises:
a passive rectification mode that operates during a high impedance modulation state when a voltage swing of the antenna is high; and
an active rectification mode that operates during a low impedance modulation state when a voltage swing of the antenna is low.

10. A system as recited in claim 9, wherein the active rectification mode is applied with an active rectifier having synchronous switches with small voltage drops.

11. A system as recited in claim 9, wherein the passive rectification mode is applied with a passive rectifier that drops a higher voltage across a plurality of diode-connected transistors.

12. A system as recited in claim 6, wherein the IC comprises a shunt-load modulation switch to back-scatter the signal.

13. A system as recited in claim 12, wherein the IC further comprises a Miller encoder to Miller-encode the continuous data stream prior to backscattering.

14. A wireless μECoG device, comprising:
a micro-fabricated, ECoG array of electrodes configured to be implanted at a brain surface to acquire neural signals;
an application-specific integrated circuit (IC) coupled to the array of electrodes and configured to digitize a voltage present on the array of electrodes; and
an antenna coupled to the IC;
wherein the IC and antenna are configured to wirelessly transmit a continuous stream of data with an external reader by electromagnetic field backscattering of a signal comprising said data;
wherein said continuous stream of data is associated with acquired neural signals from the array of electrodes; and
wherein the IC and antenna are configured to be simultaneously and continuously powered by the external reader while wirelessly transmitting the continuous stream of data with the external reader.

15. A device as recited in claim 14, wherein the array of electrodes and antenna are disposed on a flexible substrate, and have a total thickness of 1 μm to 100 μm.

16. A device as recited in claim 15, wherein the array of electrodes and antenna are sufficiently flexible to conform to a highly folded cortical surface.

17. A device as recited in claim 14, wherein the IC comprises a dual-mode rectifier to maintain a constant output power to the IC and array of electrodes while modulating the continuous stream of data for transmission.

18. A device as recited in claim 17, wherein the dual-mode rectifier comprises a passive rectifier and an active rectifier connected in parallel;
wherein the passive rectifier comprises a high-impedance rectifier that is activated when a data modulated impedance is switched to high impedance; and
wherein the active rectifier comprises a low impedance active rectifier that is activated when the data modulated impedance is switched to low impedance.

19. A device as recited in claim 17, wherein the dual-mode rectifier comprises:
a passive rectification mode that operates during a high impedance modulation state when a voltage swing of the antenna is high; and
an active rectification mode that operates during a low impedance modulation state when a voltage swing of the antenna is low.

20. A device as recited in claim 19, wherein the active rectification mode is applied with an active rectifier having synchronous switches with small voltage drops.

21. A device as recited in claim 19, wherein the passive rectification mode is applied with a passive rectifier that drops a higher voltage across a plurality of diode-connected transistors.

22. A method for wirelessly transmitting μECoG signal across a tissue, comprising:
implanting an ECoG array of electrodes at a brain surface;
wirelessly digitizing a voltage present on the array of electrodes;
acquiring continuous stream of data corresponding to neural signals from the array of electrodes; and
backscattering a signal comprising said continuous stream of acquired data and wirelessly transmitting said signal to an external reader;
wherein the implant is simultaneously and continuously powered by the external reader while wirelessly transmitting the continuous stream of data with the external reader.

23. A method as recited in claim 22, wherein the array of electrodes are disposed on a substrate with an antenna, and are sufficiently flexible to conform to a highly folded cortical surface of the brain surface.

24. A method as recited in claim 22, wherein a constant output power is maintained to array of electrodes while modulating the continuous stream of data for transmission.

25. A method as recited in claim 24, wherein the continuous stream of data and power transmission are modulated via a dual-mode rectifier.

26. A method as recited in claim 24, wherein the dual-mode rectifier comprises:
a passive rectification mode that operates during a high impedance modulation state when a voltage swing of the antenna is high; and
an active rectification mode that operates during a low impedance modulation state when a voltage swing of the antenna is low.

27. A method as recited in claim 26, wherein the active rectification mode is applied with an active rectifier having synchronous switches with small voltage drops.

28. A method as recited in claim 26, wherein the passive rectification mode is applied with a passive rectifier that drops a higher voltage across a plurality of diode-connected transistors.

29. A method as recited in claim 26, further comprising Miller-encoding the continuous data stream prior to backscattering.
